(19) United States
(12) Patent Application Publication
(10) Pub. No.: US 2010/0255541 A1
(43) Pub. Date: Oct. 7, 2010

(54) Advanced Algal Photosynthesis-Driven Bioremediation Coupled with Renewable Biomass and Bioenergy Production

(75) Inventors: Qiang Hu, Chandler, AZ (US); Milton Sommerfeld, Chandler, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Arizona Board of Regents, a body corporate acting for and on behalf of Arizona State University, Scottsdale, AZ (US)

(21) Appl. No.: 12/600,157

(22) PCT Filed: May 16, 2008
(86) PCT No.: PCT/US08/64009

§ 371 Date: Mar. 8, 2010

Related U.S. Application Data
(60) Provisional application No. 60/930,381, filed on May 16, 2007.
(60) Provisional application No. 60/930,359, filed on May 16, 2007.
(60) Provisional application No. 60/930,380, filed on May 16, 2007.
(60) Provisional application No. 60/930,379, filed on May 16, 2007.
(60) Provisional application No. 60/930,454, filed on May 16, 2007.

(51) Int. Cl. C12P 21/00; C12N 1/12; C12M 1/00; C02F 3/32

(52) U.S. Cl. 435/71.1; 435/257.1; 435/292.1; 435/257.5; 210/602

(57) ABSTRACT

The present invention relates to algal species and compositions, methods for identifying algae that produce high lipid content, possess tolerance to high CO₂, and/or can grow in waste streams, and methods for using such algae for waste stream remediation and biomass production.

### RELATED APPLICATIONS

This application claims priority to U.S. Provisional Patent Application Ser. Nos. 60/930,359 filed May 16, 2007; No. 60/930,380 filed May 16, 2007; No. 60/930,381 filed May 16, 2007; No. 60/930,379 filed May 16, 2007; and No. 60/930,454 filed May 16, 2007; all of which are incorporated by reference herein in their entirety.

### FIELD

The invention relates to algae, algae selection methods, and methods for using algae to remediate waste streams and make various products.

### BACKGROUND

The two greatest challenges facing the world in the twenty-first century are environmental degradation and a identifying a sustainable energy source. Global warming due to increases in CO₂ and other greenhouse gases (methane, chloroflurocarbons, etc.) in the atmosphere, and widespread water pollution with nutrients (such as nitrogen and phosphate) and other contaminants, are the major environmental concerns. Although many conventional techniques and approaches are available for pollution prevention and control, these methods are usually very expensive with high energy consumption. Large quantities of sludge and/or liquid wastes generated from these systems are difficult to deal with and may also pose the risk of creating secondary contamination. Oil, natural gas, coal, and nuclear energy are the predominant sources of energy used today and they are not sustainable. As energy consumption increases, the natural reserves of these nonrenewable fossil fuels shrink drastically. For instance, at the current rate of consumption, currently identified oil reserves will last approximately 50 years or less. Production and consumption of fossil fuels are also the major causes of regional and global air and water pollutions. Therefore, development and implementation of diverse, renewable, sustainable energy sources becomes increasingly important.

Methods and reagents that can effectively remove nutrients from wastestreams while simultaneously producing high oil-containing feedstock for biodiesel production, and other value-added biomass which can be used, for example, as animal feed and organic fertilizer, would be a great benefit to the art. An engineered bacterial system may be designed that can breakdown and remove nutrients and other contaminants from waste streams, but it can not effectively convert and recycle waste nutrients into renewable biomass. Many oil crops such as soy, rapeseeds, sunflower seeds, and palm seeds are a source of feedstock for biodiesel, but these crops cannot adequately perform wastestream treatment.

### SUMMARY

In an embodiment, an isolated Chlorococcum species is provided that is characterized by (i) an optimal growth temperature over 40° C., (ii) the ability to grow in a high CO₂ environment, (iii) an ability to accumulate large quantities of lutein, and (iv) an ability to assimilate large quantities of nutrients selected from the group consisting of nitrogen, phosphorous, and inorganic carbon, or progeny thereof.

In an embodiment, an isolated Chlorococcum species deposited under ATCC Accession No. ______, and mutant strains derived therefrom.

In an embodiment, an isolated Scenedesmus species is provided that is characterized by an ability to grow in a high CO₂ environment, and an ability to accumulate carotenoids selected from the group consisting of lutein, zeaxanthin, and astaxanthin, or progeny thereof.

In an embodiment, an isolated Scenedesmus species deposited under ATCC Accession No. ______, and mutant strains derived therefrom.

In an embodiment, an isolated Palmellococcus species is provided that is characterized by an ability to grow in a high CO₂ environment, and an ability to accumulate astacene, or progeny thereof.

In an embodiment, an isolated Palmellococcus species deposited under ATCC Accession No. ______, and mutant strains derived therefrom.

In an embodiment, an isolated Cylindrospermopsis species is provided that is characterized by an ability to assimilate large quantities of nutrients selected from the group consisting of nitrogen, phosphorous, and inorganic carbon, as well as an ability to accumulate large quantities of protein mass, and an ability to accumulate phycobiliproteins selected from the group consisting of phycocyanin, allophycocyanin, and phycoerythrin, or progeny thereof.

In an embodiment, an isolated Cylindrospermopsis species deposited under ATCC Accession No. ______, and mutant strains derived therefrom.

In an embodiment, an isolated Planktothrix species is provided that is characterized by an ability to assimilate large quantities of nutrients selected from the group consisting of nitrogen, phosphorous, and inorganic carbon, an ability to accumulate large quantities of protein mass, and an ability to accumulate phycobiliproteins selected from the group consisting of phycocyanin, allophycocyanin, and phycoerythrin, or progeny thereof.

In an embodiment, an isolated Planktothrix species deposited under ATCC Accession No. ______, and mutant strains derived therefrom.

In another embodiment, a substantially pure culture, including a growth medium, and an isolated organism, are provided.

In other embodiments, a system, including a photobioreactor; and a substantially pure culture of an organism, are also provided.

In other embodiments, methods are provided for removing nutrients from wastestreams, including adding a wastestream to the substantially pure culture of embodiments of the disclosure, whereby nutrients in the wastestream are removed by the algae present in the culture.

In other embodiments, methods are provided for producing biomass, including culturing the algae of embodiments of the disclosure and harvesting algal protein and/or biomass components from the cultured algae.

In another embodiment, methods are provided for simultaneously removing nutrients from wastestreams and producing biomass, including adding a waste stream to the substantially pure culture of any of the above embodiments, whereby nutrients in the waste stream are removed by the algae present in the culture; and harvesting algal protein and/or biomass components.

### DETAILED DESCRIPTION

In one aspect, an isolated Chlorococcum species is provided that is characterized by (i) an optimal growth temperature over 40° C., (ii) the ability to grow in a high CO₂ environment, (iii) an ability to accumulate large quantities of lutein, and (iv) an ability to assimilate large quantities of nutrients selected from the group consisting of nitrogen, phosphorous, and inorganic carbon, or progeny thereof.

In another aspect, an isolated Scenedesmus species is provided that is characterized by (i) an ability to grow in a high CO₂ environment, and (ii) an ability to accumulate carotenoids selected from the group consisting of lutein, zeaxanthin, and astaxanthin, or progeny thereof.

In another aspect, an isolated Palmellococcus species is provided that is characterized by (i) an ability to grow in a high CO₂ environment, and (ii) an ability to accumulate astacene, or progeny thereof.

In one aspect, an isolated Cylindrospermopsis species is provided that is characterized by (i) an ability to assimilate large quantities of nutrients selected from the group consisting of nitrogen, phosphorous, and inorganic carbon, (ii) an ability to accumulate large quantities of protein mass, and (iii) an ability to accumulate phycobiliproteins selected from the group consisting of phycocyanin, allophycocyanin, and phycoerythrin), or progeny thereof.

In one aspect, an isolated Planktothrix species is provided that is characterized by (i) an ability to assimilate large quantities of nutrients selected from the group consisting of nitrogen, phosphorous, and inorganic carbon, (ii) an ability to accumulate large quantities of protein mass, and (iii) an ability to accumulate phycobiliproteins selected from the group consisting of phycocyanin, allophycocyanin, and phycoerythrin, or progeny thereof.

In some embodiments, the algae of the present disclosure can effectively remove nutrients from wastestreams while simultaneously producing high oil-containing feedstock for biodiesel production, and other value-added biomass which can be used, for example, as animal feed and organic fertilizer.

As used herein, the term “algae” includes both microalgae and cyanobacteria, and the algae of the disclosure include any strain with the identifying characteristics described above, and any progeny derived from such strains.

As used herein the term “isolated” means that at least 90% of the microorganisms present in the isolated algae composition are of the recited algal type; more preferably at least 95%, even more preferably at least 98%, and even more preferably 99% or more.

The isolated algae can be cultured or stored in solution, frozen, dried, or on solid agar plates.

As used herein, the phrase “ability to grow” means that the algae are capable of reproduction under the recited conditions.

As used herein, the phrase “ability to accumulate large quantities” means the following: for long-chain polyunsaturated fatty acids (such as EPA, DHA, ALA, and GLA) and high-value carotenoids (such as beta-carotene, zeaxanthin, luteine, astaxanthin), large quantities mean, for example, 0.5 to 6% of cell dry weight. For phycobiliproteins, which are another group of water soluble photosynthetic pigments in cyanobacteria and red algae, large quantities mean 4 to 16% of dry weight. In the case of crude proteins, total lipids, or total polysaccharides, the phrase “large quantities” means 20 to 60% of dry weight.

As used herein, the phrase “an ability to assimilate large quantities of nutrients” means the following: for nitrogen (nitrate or ammonia/ammonium) removal from contaminated water and wastewater, 2-4 mg per liter of nitrogen as nitrate or ammonia per hour of treatment is regarded as a high removal rate (i.e. assimilating large quantities of nutrients). In the case of CO₂ removal from power plant flue gas emissions, 2 to 4 grams of CO₂ per liter of algal culture per hour of cultivation time is regarded as a high removal rate.

In one embodiment, the isolated algae is a high temperature-tolerant Chlorococcum mutant (Chlorophyceae) that has the ability to thrive at culture temperatures ranging from 10° C. to 48° C. with an optimal growth temperature over 40° C. This mutant can thrive at high levels of carbon dioxide (10 to 20% dissolved CO₂/air; i.e. dissolved CO₂ in a culture medium the algae grow in). Few algal species/strains have the ability to thrive at elevated CO₂ concentrations much higher than 10% of CO₂ in air. The exact toxicity of high levels of CO₂ to algae is poorly understood, but may exert two separate impacts on algal survival and proliferation: 1) high concentration of CO₂ itself may have negative effects, and high CO₂-induced low pH effects. It also has the ability to synthesize and accumulate large quantities of a high-value carotenoid, lutein, while rapidly taking up and assimilating nutrients (e.g., nitrogen, phosphorous, inorganic carbon) from water and wastewater from various sources.

Mutagenesis and isolation of algal mutants was performed as follows: chemical mutagenesis of microalgae was performed using the chemical mutagen, N-methyl-N′-nitro-N-nitrosoguanidine (MNNG). Briefly, Chlorococcum cells in the exponential growth phase were incubated with 50 lag MNNG mL-1 at 25° C. for 30 min. Mutagenesis was terminated by adding an equal volume of freshly made 10% (w/v) filter-sterilized sodium thiosulfate into the reaction solution. Treated cells were collected by centrifugation (2,000×g, 25° C., 10 min). For expression of mutations, the mutagenized cells were incubated on agar plates containing the acetate basal medium and 20 mg/mL ampicillin (sodium salt). When mutagenized colonies developed on the agar plate, they were transferred individually into test tubes containing 5 mL of liquid acetate basal medium and incubated in a growth chamber at 22° C. and 20 umol m⁻²s⁻¹ of light under the light/dark cycle of 12 h.

Isolated mutants were screened for specific phenotypic traits. These traits included, but were not limited to, the ability to produce and accumulate high concentrations of specific compounds such as lipids/fatty acids and/or carotenoids, and/or exhibit high growth (i.e. one to two cell doubling time per day or three to four doubling time per 24 hour (in case of indoor culture under continuous illumination) are regarded as high growth rate), and nutrient uptake potential, and/or exert greater tolerance to a broader range of environmental and culture conditions such as light intensity (200-2000 umol m⁻²s⁻¹), temperature (15° C. to 40° C.), CO₂ concentration (1 to 20% CO₂/air), ammonia/ammonium concentrations (400-1,000 mg L-1 nitrogen), salinity (½, 1, 2, and 3 times of sea water), or culture pH (pH 5 to 10).

In a further embodiment of the disclosure, a green alga Scenedesmus sp. is disclosed. This strain was isolated from a unique natural aquatic habitat where dissolved CO₂ concentrations were nearly 600 times higher than that commonly occurs in freshwater (−0.31 ml L⁻¹). The ability to survive at high CO₂ environment makes this algal strain extremely suitable for biological sequestration of CO₂ from flue gases emitted from power generators. This algal strain can also accumulate high concentrations of secondary carotenoids (e.g., lutein, zeaxanthin, and astaxanthin) under various culture conditions (such as nutrient starvation (such as nitrogen, phosphorus, iron, and/or silicon), high light intensity (200 to 2,000 umol m⁻²s⁻¹), and/or adverse temperature (below 15° C. and above 40° C.).

In some embodiments, an isolated Palmellococcus species is provided that is characterized by (i) an ability to grow in a high CO₂ environment, and (ii) an ability to accumulate astacene, or progeny thereof.

In another embodiment, a new green algal strain, Palmellococcus sp. is disclosed. This algal strain can thrive at up to 20% CO₂/air and can be used as an ideal candidate for carbon sequestration and renewable biomass production. The algal strain can also synthesize and accumulate large quantities of a novel red carotenoids astacene under stress conditions. Astacene, like astaxanthin, possesses strong antioxidant activities and provides desirable coloration of cultured salmon or other aquatic animals.

In one aspect, an isolated Cylindrospennopsis species is provided that is characterized by (i) an ability to assimilate large quantities of nutrients selected from the group consisting of nitrogen, phosphorous, and inorganic carbon, (ii) an ability to accumulate large quantities of protein mass, and (iii) an ability to accumulate phycobiliproteins selected from the group consisting of phycocyanin, allophycocyanin, and phycoerythrin, or progeny thereof.

In a further embodiment of this aspect, a planktonic, filamentous cyanobacterium Cylindrospermopsis sp is disclosed. This cyanobacterial strain was isolated from a local lake in the metro Phoenix area and exhibits rapid growth and nutrient uptake rate in nutrient-rich water and wastewater. While assimilating waste nutrients, the isolate has the ability to accumulate large quantities of proteins (up to 60% dry weight) and high-value pigments, phycobiliproteins (4 to 16% of dry weight) (include phycocyanin, allophycocyanin, and phycoerythrin).

In one aspect, an isolated Planktothrix species is provided that is characterized by (i) an ability to assimilate large quantities of nutrients selected from the group consisting of nitrogen, phosphorous, and inorganic carbon, (ii) an ability to accumulate large quantities of protein mass, and (iii) an ability to accumulate phycobiliproteins selected from the group consisting of phycocyanin, allophycocyanin, and phycoerythrin, or progeny thereof.

In another embodiment of this aspect, a planktonic, filamentous cyanobacterium Planktothrix sp is disclosed. This cyanobacterial strain was also isolated from a local lake in the metro Phoenix region and exhibits rapid growth and nutrient uptake rate in nutrient-rich water and wastewater. While assimilating waste nutrients, the isolate has the ability to accumulate large quantities of proteins (up to 55% dry weight) and high-value pigments, phycobiliproteins (up to 16% dry weight) (include phycocyanin, allophycocyanin, and phycoerythrin).

In another aspect, a substantially pure culture is provided that comprises:

a growth medium; and

an isolated organism according to an aspect of the present disclosure.

As used herein the term “isolated organism” means that at least 90% of the microorganisms present in the isolated algae composition are of the recited algal type; more preferably at least 95%, even more preferably at least 98%, and even more preferably 99% or more.

As used herein, the term “growth medium” refers to any suitable medium for cultivating algae of the present disclosure. The algae of the disclosure can grow photosynthetically on CO₂ and sunlight, plus a minimum amount of trace nutrients. The volume of growth medium can be any volume suitable for cultivation of the algae for any purpose, whether for standard laboratory cultivation, to large scale cultivation for use in, for example, bioremediation and/or algal biomass production.

For maintenance and storage purposes, individual algal isolates are usually maintained in standard artificial growth medium. For the regular maintenance purpose, the algal isolates are kept in both liquid cultures and solid agar plates under either continuous illumination or a light/dark cycle of moderate ranges of light intensities (10 to 40 umol m⁻²s⁻¹) and temperatures (18° C. to 25° C.). The culture pH may vary from pH 6.5 to pH 8.5. No CO₂ enrichment is required for maintenance of algal strains. In a non-limiting example, the temperature of culture medium in growth tanks is preferably maintained at from about 15° C. to about 38° C., more preferably between about 20° C. to about 30° C.

The pH of the culture medium is maintained at between about pH 6.5 to about pH 9.5 for optimum growth and health of the algae. It is preferable to maintain the culture within this pH. However a limited number of algae that can survive at extremely low (pH <2) or extremely high pH (pH >10), most of algal strains have a pH tolerance from 6.5 to 9.5.

A preferred growth medium useful for culturing algae of the present disclosure is prepared from wastewater or waste gases. This growth medium is particularly useful when the algae of the present disclosure are used in a waste remediation process, although use of this growth medium is not limited to waste remediation processes. In this embodiment, when wastewater is used to prepare the medium, preferably, it is preferably from nutrient-contaminated water or wastewater (e.g., industrial wastewater, agricultural wastewater domestic wastewater, contaminated groundwater and surface water), or waste gases emitted from power generators burning natural gas or biogas, and flue gas emissions from fossil fuel fired power plants.

In this preferred embodiment, the algae can be first cultivated in a primary growth medium, followed by addition of wastewater and/or waste gas. Alternatively, the algae can be cultivated solely in the wastestream source. When a particular nutrient or element is added into the culture medium, it will be up-taken and assimilated by the cells, just like the cell taking other nutrients. In the end, both wastewater-containing and spiked nutrients will be removed and converted into macromolecules (such as lipids, proteins, or carbohydrates) stored in algal biomass. Typically, the waste water is added to the culture medium at a desired rate. This water, being supplied from the waste water source, contains additional nutrients, such as phosphates, and/or trace elements (such as iron, zinc), which supplement the growth of the algae. In one embodiment, if the waste water being treated contains sufficient nutrients to sustain the microalgal growth, it may be possible to use less of the growth medium. As the waste water becomes cleaner due to algal treatment, the amount of growth medium can be increased.

The major factors affecting waste-stream feeding rate include: 1) algal growth rate, 2) light intensity, 4) culture temperature, 5) initial nutrient concentrations in wastewater; 5) the specific uptake rate of certain nutrient/s; 6) design and performance of a specific bioreactor and 7) specific maintenance protocols.

In another aspect, a system is provided that comprises:

(a) a photobioreactor; and

(b) a substantially pure culture according to an aspect of the disclosure.

As used herein, a “photobioreactor” is an industrial-scale culture vessel in which algae grow and proliferate. For use in this aspect of the disclosure, any type of photobioreactor can be used, including but not limited to open raceways (i.e. shallow ponds (water level ca. 15 to 30 cm high) each covering an area of 1000 to 5000 m² constructed as a loop in which the culture is circulated by a paddle-wheel (Richmond, 1986)), closed systems, i.e. photobioreactors made of transparent tubes or containers in which the culture is mixed by either a pump or air bubbling (Lee 1986; Chaumont 1993; Richmond 1990; Tredici 2004), tubular photobioreactors (For example, see Tamiya et al. (1953), Pirt et al. (1983), Gudin and Chaumont 1983, Chaumont et al. 1988; Richmond et al. 1993)) and flat plate-type photobioreactors, such as those described in Samson and Leduy (1985), Ramos de Ortega and Roux (1986), Tredici et al. (1991, 1997) and Hu et al. (1996, 1998a,b).

The distance between the sides of a closed photobioreactor is the “light path,” which affects sustainable algal concentration, photosynthetic efficiency, and biomass productivity. In various embodiments, the light path of a closed photobioreactor can be between approximately 5 millimeters and 40 centimeters; between 100 millimeters and 30 centimeters, between 50 millimeters and 20 centimeters, and between 1 centimeter and 15 centimeters, and most preferably between 2 centimeters and 10 centimeters. The most optimal light path for a given application will depend, at least in part, on factors including the specific algal strains to be grown and/or specific desired product/s to be produced.

In this aspect, systems of various designs are provided that can be used, for example, in methods for nutrient removal (described below) using algal strains according to aspects of the disclosure.

In another aspect, methods are provided for removing nutrients from wastestreams, comprising adding a waste stream to the substantially pure culture of aspects of the disclosure, whereby nutrients in the waste stream are removed by the algae present in the culture. Through this process up to 95% or more of the nutrients will be removed from the water or wastewater, resulting in nutrient levels below maximum contaminant levels set for individual contaminants by the US EPA.

As used herein, the term “wastestream” refers to any high nutrient containing (e.g., nitrogen, phosphate, and/or CO₂)stream of fluid, such as wastewater or waste gas. One non-limiting example of such wastestreams is groundwater that may contain tens or hundreds of milligrams per liter of nitrogen as nitrate. The amounts of nitrate can be removed to below 10 mg nitrate-per liter within one or several days, depending on initial nitrate concentration in the groundwater. The amount of groundwater that can be purified by this method depends on the initial concentrations of nutrient's to be removed and the size of bioreactor system used. In some cases, the groundwater may be spiked with trace amounts of phosphate (in a range of micro- or milligrams per liter) or microelements (such as Zn, Fe, Mn, Mg) in order to enable the algae to completely remove nitrate from the groundwater.

In another non-limiting embodiment, wastewater can come from Concentrated Animal Feeding Operations (CAFOs), such as dairy farms, which may contain high concentrations of ammonia (hundreds to thousands of milligrams per liter of nitrogen as ammonia) and phosphate (tens to hundreds of milligrams per liter of phosphorous as phosphate). Full-strength CAFO wastewater can be used as a “balanced growth medium” for sustaining rapid growth of selected algal strains in photobioreactors of aspects of the disclosure. In some cases the CAFO wastewater can be diluted to a certain extent to accelerate growth and proliferation of algal strains. As a result, ammonia and phosphate concentrations can be removed with one or several days, depending on initial concentrations of these nutrients. In contrast to the groundwater situation, no chemicals are required to be introduced into CAFO wastewater in order to reduce or eliminate ammonia and phosphate levels to meet the US EPA standards.

In another embodiment, wastewater is agricultural runoff water that may contain high concentrations (in a range of several to tens of milligrams per liter) of nitrogen in forms of nitrate and ammonia and phosphates. The algae of the present disclosure can remove these nutrients to below the US EPA's standards within one day or two, depending on initial concentrations of these nutrients and/or weather conditions. In case the nitrogen to phosphorous ratio is distant from the ratio of 15:1, addition of one chemical (either nitrates or phosphates) to balance the ratio is necessary to remove these nutrients from the wastewater.

In another embodiment of this aspect, the waste stream comprises flue gas emissions as a carbon source (in a form of carbon dioxide, or CO₂) for algal photosynthesis and waste nutrient removal. Flue gases may be those from any source, including but not limited to fossil fuel-burning power plants. Through the photosynthetic machinery, algal cells fix CO₂ and convert it into organic macromolecules (such as carbohydrates, lipids, and proteins) stored in the cell. As a result, molecular CO₂ entering into the culture system disclosed above is removed and converted into algal biomass, and thus the gas released from the photobioreactor will be significantly reduced in CO₂ (at least a 75% reduction).

In one embodiment, flue gases are delivered into the photobioreactor disclosed above. One method involves injection of the flue gas directly into the photobioreactor at a flow rate to sustain (0.1 to 0.5 liter of flue gas per liter of culture volume per minute) vigorous photosynthetic CO₂ fixation while exerting minimum negative effects due to lowering culture pH by dissolved NO, SO, and/or certain toxic molecules such as the heavy metal mercury. Alternatively, the flue gas may be blended with compressed air at a certain ratio (flue gas to compressed air ratio may range from 0.1-0.6 volume to 1 volume) and delivered into the photobioreactor through an aeration system. In a preferred embodiment, a liquid- or gas-scrubber system may be introduced to reduce or eliminate contaminant transfer from the gas-phase and accumulation of toxic compounds in the algal growth medium. In a further preferred embodiment, flue gases coming out from the power generator may be pre-treated with proton-absorbing chemicals such as NaOH to maintain an essentially neutral pH and turn potentially harmful NO and SO compounds into useful sulfur and nitrogen sources for algal growth. For example, a commercially available gas-scrubber can be incorporated into the photobioreactor system to provide algae with pretreated flue gas. In case of liquid wastes, pre-treatment can include but is not limited to 1) wastewater treated first through an anaerobic digestion process or natural or constructed wetland to remove most of organic matters; 2) dilute wastewater 10 to 90% dilution with regular ground or surface water, depending on concentrations of potential toxic compounds; 3) addition of certain nutrients (such as phosphorous and/or trace elements) to balance the nutrient composition for maximum sustainable nutrient removal and/or biomass production.

In another aspect, methods for producing biomass are provided that comprise culturing the algae of an aspect of the disclosure and harvesting algal protein and/or biomass components from the cultured algae. In one embodiment, a multistage maintenance protocol is described to remove waste nutrients at the early stages, while inducing and accumulating high-value compounds (such as lipids/oil, carotenoids) at later stages. In a preferred embodiment, algal biomass produced from the photobioreactor will be used as feedstock for biodiesel production. In a further preferred embodiment, residues of algal mass after extraction of algal oil/lipids will be used as animal feed or organic fertilizer additive. In another embodiment, carotenoid-rich algal biomass as a by-product of waste-stream treatment by algal strains grown in the photobioreactors described above is used as an animal feed additive or a natural source of high-value carotenoids. Methods for algal biomass production and/or protein expression are well known in the art. See, for example:

Hu, Q. (2004) Chapter 5: Environmental effects on cell composition, pp. 83-93. In Richmond A. (ed.) Handbook of Microalgal Culture, Blackwell Science Ltd, Oxford OX2 OEL, UK.

Hu, Q. (2004) Chapter 12: Industrial production of microalgal cell-mass and secondary products Major industrial species: Arthrospira (Spirulina) platensis, pp. 264-272. In Richmond A. (ed.) Handbook of Microalgal Culture, Blackwell Science Ltd, Oxford OX2 OEL, UK.

Hu, Q., Westerhoff, P. and Vermaas, W. (2000) Removal of nitrate from drinking water by cyanobacteria: quantitative assessment of factors influencing nitrate uptake. Appl. Env. Microbiol. 66: 133-139.

Hu, Q., Marquardt, J., Iwasaki, I., Miyashita, H., Kurano, N., MOrschel, E. and Miyachi, S. (1999) Structure, localization and function of biliproteins from the chlorophyll a/d containing prokaryote, Acaryochloris marina. Biochim. Biophys. Acta, 1412: 250-261.

Hu, Q., Miyashita, H., Iwasaki, I., Miyachi, S., Iwaki, M. and Itoh, S. (1998) A photosystem I reaction center driven by chlorophyll d in oxygenic photosynthesis. Proc. Natl. Acad. Sci. USA, 95: 13319-13323.

Hu, Q., Ishikawa, T., Inoue, Y., Iwasaki, I., Miyashita, H., Kurano, N., Miyachi, S., Iwaki, M. and Itoh, S. (1998) Heterogeneity of chlorophyll d-binding photosystem I reaction centers from the photosynthetic prokaryote Acaryochloris marina. In: Garab G. (ed.) Photosynthesis: Mechanisms and Effects, Vol. I. 437-440, Kluwer Academic Publishers, Dordrecht, The Netherlands.

Hu., Q., Faiman, D. and Richmond, A. (1998) Optimal orientation of enclosed reactors for growing photoautotrophic microorganisms outdoors. J. Ferment. Biotechnol. 85: 230-236.

Hu Q., Yair, Z. and Richmond, A. (1998) Combined effects of light intensity, light-path and culture density on output rate of Spirulina platensis (Cyanobacteria). Eur. J. 40 Phycol. 33: 165-171.

Hu Q., Kurano, N., Iwasaki, I., Kawachi, M. and Miyachi, S. (1998) Ultrahigh cell density culture of a marine green alga, Chlorococcum littorale in a flat plate photobioreactor. Appl. Microbiol. Biotechnol. 49: 655-662.

Iwasaki, I., Hu Q., Kurano, N. and Miyachi, S. (1988) Effect of extremely high-CO₂ stress on energy distribution between photosystem I and photosystem II in a ‘HighCO₂’ tolerant green alga, Chlorococcum littorale and the intolerant green alga Stichococcus bacillaris. J. Photochem. Photobiol. B: Biology 44: 184-190.

Hu Q., Hu, Z., Cohen, Z. and Richmond, A. (1997) Enhancement of eicosapentaenoic acid (EPA) and y-linolenic acid (GLA) production by manipulating algal density of outdoor cultures of Monodus subterraneus (Eustigmatophyte) and Spirulina platensis (Cyanobacterium). Eur. J. Phycol. 32: 81-86.

Richmond, A. and Hu, Q. (1997) Principles for utilization of light for mass production of photoautotrophic microorganisms. Appl. Biochem. Biotechnol. 63-65: 649-658.

Hu Q., Guterman, H. and Richmond, A. (1996) A flat inclined modular photobioreactor (FIMP) for outdoor mass cultivation of photoautotrophs. Biotechnol. Bioeng. 51: 51-60.

Hu Q., Guterman, H. and Richmond, A. (1996) Physiological characteristics of Spirulina platensis cultured at ultrahigh cell densities. J. Phycol. 32: 1066-1073.

Hu, Q. and Richmond, A. (1996) Productivity and photosynthetic efficiency of Spirulina platensis affected by light intensity, cell density and rate of mixing in a flat plate photobioreactor. J. Appl. Phycol. 8: 139-145.

Gitelson, A., Hu, Q. and Richmond, A. (1996) Photic volume in photobioreactors supporting ultrahigh population densities of the photoautotroph Spirulina platensis. Appl. Env. Microbiol. 62: 1570-1573.

Hu, Q. and Richmond, A. (1995) Interrelationships between the photoinhibition, photolimitation of photosynthesis and biomass productivity: Effect of population density. In: Mathis P. (ed.) Photosynthesis: from Light to Biosphere, Vol. IV, 10371040, Kluwer Academic Publishers, The Netherlands.

Hu, Q. and Richmond, A. (1994) Optimizing the population density of Isochrysis galbana grown outdoors in a glass column photobioreactor. J. Appl. Phycol. 6: 391-396.

In another aspect, methods are provided for simultaneously removing nutrients from wastestreams and producing biomass, comprising: adding a waste stream to the substantially pure algal culture of aspects of the disclosure, whereby nutrients in the waste stream are removed by the algae present in the culture; and harvesting algal protein and/or biomass components.

Embodiments of the present disclosure address environmental pollution control while producing renewable energy through novel algal reagents and methods. Algae of the disclosure are used to rapidly remove nutrients from wastestreams (including but not limited to wastewater and power plant flue gases) and convert them into value-added compounds stored into algal biomass. The biomass can then be used, for example, as feedstock for production of liquid biofuel and/or fine chemicals, and used as animal feed, or organic fertilizer. The major advantages of reagents and methods of the present disclosure over conventional bacteria-based systems are that it not only removes nutrients from wastestreams, but also recycles them in form of renewable biomass and fine chemicals, whereas bacterial systems strip off potentially valuable nitrate and/or ammonia into the atmosphere through nitrification and denitrification processes. Bacterial systems also usually generate large amounts of sludge which require proper disposal. Compared to natural and constructed wetland systems, the algae-based reagents and methods of the present disclosure are more efficient in terms of nutrient removal and biomass production.

From the energy production side, the reagents and methods of the present disclosure are more efficient than conventional oil crop production, producing up to 20 to 40 times more feedstock per unit area of land per year. The reagents and methods of the present disclosure can be applied in non-agricultural environments, such as arid and semi-arid environments (including deserts). Thus, the present technology will not compete with oilseeds (or other) plants for limited agricultural land. Algal feedstock produced by the methods of the disclosure can be used for purposes including, but not limited to, biodiesel production.

### EXAMPLES

Optical Density and Dry Weight Measurements:

Algal cell population density is measured daily using a micro-plate spectrophotometer (SPECTRA max 340 PC) and reported as optical density at 660 nm wave length. The dry weight of algal mass is determined by filtration from 10-20 ml culture through a pre-weighed Whatman GF/C filter. The filter with algae is dried at 105° C. overnight and cooled to the room temperature in a desiccator and weighed.

Chlorophyll Measurement:

A hot methanol extraction method is used (Azov (1982). The concentration is calculated using the Tailing coefficient:

Chlorophyll a (mg/L)=13.9 (DO₆₆₅-DO₇₅₀) V/U where DO₆₆₅₌optical density measured at 665 nm wavelength, DO750=optical density measured at 750 nm wavelength, V=total volume of methanol (ml), and U=volume of algal suspension (ml).

Lipid Extraction: The lipid extraction procedure is modified according to Bigogno et al. (2002).

Algal cell biomass (100 mg freeze-dried) is added to a small glass vial sealed with Teflon screw cap and is extracted with methanol containing 10% DMSO, by warming to 40° C. for 1 hour with magnetic stirring. The mixture is centrifuged at 3,500 rpm for ten minutes. The resulting supernatant is removed to another clean vial and the pellet is re-extracted with a mixture of hexane and ether (1:1, v/v) for 30 minutes. The extraction procedure is repeated several times until negligible amounts of chlorophylls remain in the pellet. Diethyl ether, hexane and water are added to the combined supernatants, so as to form a ratio of 1:1:1:1 (v/v/v/v). The mixture is hand-shaken and then centrifuged at 3,500 rpm for 5 minutes. The upper phase is collected. The lower water phase is re-extracted twice with a mixture of diethyl ether:hexane (1:1, v/v). The organic phases are combined, and the solvents in the oil extract are completely removed by bubbling with nitrogen gas until the weight of the remaining oil extract is constant.

Fatty Acid Analysis:

Fatty acids are analyzed by gas chromatography (GC) after direct transmethylation with sulphuric acid in methanol (Christie, 2003). The fatty acid methanol esters (FAMEs) are extracted with hexane containing 0.8% BHT and analyzed by a HP-6890 gas chromatography (Hewlett-Packard) equipped with HP7673 injector, a flame-ionization detector, and a HP-INNO WAX™ capillary column (HP 19091N-133, 30 m×0.25 mm×0.25 μm). Two (2) μL of the sample is injected in a split-less injection mode. The inlet and detector temperatures are kept at 250° C. and 270° C., respectively, and the oven temperature is programmed from 170° C. to 220° C. increasing at 1° C./minute. High purity nitrogen gas is used as the carrier gas. FAMEs are identified by comparison of their retention times with those of the authentic standards (Sigma), and are quantified by comparing their peak areas with that of the internal standard (C 17:0).

Collection of Dairy Wastewater:

Dairy wastewater is collected at a dairy from a shallow wastewater pond consisting of piped dairy stall waste and overland runoff. A composite wastewater sample is collected from no fewer than three access points along the bank of a shallow wastewater pond. Wastewater is stored in a plastic container (5 gallons or larger) at 4° C. Wastewater, in raw form, is brownish-red colored and contained undigested grains, grasses, soil and other unidentified solids. Before use for experiments, the dairy wastewater is centrifuged to remove particles and native species of algae at 5,000 rpm. The clear brown dairy wastewater is collected for assigned experiments. The wastewater is diluted to 25% wastewater (1:3 dairy wastewater to deionized water), 50% wastewater (1:1 wastewater to deionized water), 75% wastewater (3:1 wastewater to deionized water), and 100% wastewater (undiluted wastewater) to meet various experimental needs.

Experimental Design:

A 300-ml capacity glass column (68 cm long with an inner diameter of 2.3 cm) with a glass capillary rod placed down the center of the column to provide aeration is used to grow the alga. The top of the column is covered with a rubber stopper surrounded by loosely-fitting aluminum foil to prevent contamination among columns Unless otherwise stated, a culture temperature of 25° C., a light intensity of 170 μmol m⁻² s⁻¹, and compressed air of 1% CO₂ are applied to glass columns throughout the experiment. For experiments, log-phase cultures are harvested and centrifuged to remove the culture medium and re-suspended into a small volume of sterilized distilled water for inoculation. Each treatment is run in triplicate. Deionized water is added daily to the column to compensate for water loss due to evaporation. For nutrient removal experiments, 10 ml of culture suspension is collected from the column daily and centrifuged at 3,500 rpm for 10 minutes. The supernatant is pooled into small vials and frozen in a −20° C. freezer for nutrient analysis. The pellets are re-suspended into distilled water for dry weight measurement.

High Carbon Dioxide Treatment:

For CO₂ treatment experiments, algal cells are grown in BG-11 growth medium either bubbled with air enriched with 1% CO₂, or air enriched with 15% CO₂.

### REFERENCES

Chaumont D. (1993) Biotechnology of algal biomass production: a review of systems for outdoor mass culture. J Appl Phycol 5: 593-604

Chaumont D., Thepenier C. and Gudin C. (1988) Scaling up a tubular photobioreactor for continuous culture of Porphyridium cruentum-from laboratory to pilot plant, pp. 199-208. In Stadler T, Morillon J, Verdus MS, Karamanos W, Morvan H, Christiaen D (eds.), Algal Biotechnology. Elsevier Applied Science, London. Cornwell, D. A. (1990). Air Stripping and Aeration, in Water Quality and Treatment, McGraw Hill Inc., New York, pg. 229-268.

Fernandez, F. G., Camacho, F. G., Perez, J. A., Sevilla, J. M., Grima, E. M. (1998). Modeling of biomass productivity in tubular photobioreactors for microalgal cultures: effects of diluation rate, tube diameter, and solar irradiance, Biotech. And Bioeng., 58:6:605-616.

Gitelson A., Hu Q. and Richmond A. (1996) Photic volume in photobioreactors supporting ultrahigh population densities of the photoautotroph Spirulina platensis. Applied and Environmental Microbiology 62: 1570-1573.

Gudin C. and Chaumont D. (1991) Cell fragility—the key problem of microalgae mass production in closed photobioreactors. Bioresource Technol. 38: 145-151.

Hu Q. and Richmond A. (1994) Optimizing the population density of Isochrysis galbana grown outdoors in a glass column photobioreactor. Journal of Applied Phycology 6:391-396.

Hu Q., Faiman D. and Richmond A. (1998a) Optimal orientation of enclosed reactors for growing photoautotrophic microorganisms outdoors. Journal of Fermentation and Biotechnology 85: 230-236.

Hu Q., Guterman H. and Richmond A. (1996a) A flat inclined modular photobioreactor (FIMP) for outdoor mass cultivation of photoautotrophs. Biotechnology and Bioengineering 51: 51-60.

Hu Q., Guterman H. and Richmond A. (1996b) Physiological characteristics of

Spirulina platensis cultured at ultrahigh cell densities. Journal of Phycology 32: 1066-1073.

Hu Q., Kurano N., Iwasaki I., Kawachi M. and Miyachi S. (1998b) Ultrahigh cell density culture of a marine green alga, Chlorococcum littorale in a flat plate photobioreactor. Applied Microbiology and Biotechnology 49: 655-662.

Hu Q., Yair Z. and Richmond A. (1998c) Combined effects of light intensity, light-path and culture density on output rate of Spirulina platensis (Cyanobacteria). European Journal of Phycology 33: 165-171.

Hu Q. 2004. Industrial production of microalgal cell-mass and secondary products—major industrial species: Arthrospira (Spirulina) platensis. Pp. 264-272. In: Richmond A. (ed.) Handbook of microalgal culture: biotechnology and applied Phycology, Blackwell Science Ltd., Oxford, UK.

Iwasaki I., Hu Q., Kurano N. and Miyachi S. (1988) Effect of extremely high-0O2 stress on energy distribution between photosystem I and photosystem II in a ‘High-CO2’ tolerant green alga, Chlorococcum littorale and the intolerant green Alga Stichococcus bacillaris. Journal of Photochemistry and Photobiology B: Biology 44/3: 184-190.

Lee Y. K. (1986) Enclosed bioreactor for the mass cultivation of photosynthetic microorganism: the future trend. Trends Biotechnol. 4: 186-189.

Lee Y. K., Ding S. Y., Low C. S. and Chang Y. C. (1995) Design and performance of an a-type tubular photobioreactor for mass cultivation of microalgae. J. Appl. Phycol. 7:47-51.

Pirt S. J., Lee Y. K., Walach M. R., Pirt M. W., Balyuzi H. H. M. and Bazin M. J. (1983) A tubular bioreactor for photosynthetic production of biomass from carbon dioxide: design and performance. J. Chem. Tech. Biotechnol. 33: 35-58.

Ramos de Ortega and Roux J. C. (1986) Production of Chlorella biomass in different types of flat bioreactors in temperate zones. Biomass 10: 141-156.

Richmond A. (1990) Large scale microalgal culture and applications. In: Round/Chapman (eds) Progress in Phycological Research Biopress, London, 7: 1-62

Richmond A. (1992) Open systems for mass production of photoautotrophic microalgae outdoors: physiological principles. J. Appl. Phycol. 4: 281-286

Richmond A. and Hu Q. (1997) Principles for utilization of light for mass production of photoautotrophic microorganisms. Applied Biochemistry and Biotechnology. 63-65: 649-658.

Richmond A., Boussiba S., Vonshak A. and Kopel R. (1993) A new tubular reactor for mass production of microalgae outdoors. J. Appl. Phycol. 5: 327-332.

Samson R & Leduy A (1985) Multistage continuous cultivation of blue-green alga Spirulina maxima in the flat tank photobioreactors with recycle. Can. J. Chem. Eng. 63: 105-112.

Silva, H. J., Cortinas T. and Ertola R. J. 1987. Effect of hydrodynamic stress on Dunaliella growth. J. Chem. Tech. Biotechnol. 40: 253-264. Tamiya H. (1957) Mass culture of algae. Ann Rev. Plant Physiol. 8: 309-334.

Torzillo G., Carlozzi P., Pusharaj B., Montani E. and Materassi R. (1994) A two-plane tubular photobioreactor for outdoor culture of Spirulina. Biotechnol. Bioeng. 42: 891-898.

Tredici M. R. and Materassi R. (1992) From open ponds to vertical alveolar panels: the Italian experience in the development of reactors for the mass cultivation of photoautotrophic microorganisms. J. AppL Phycol. 4: 221-31.

Tredici M. R., Carlozzi P., Zittelli G. C. and Materassi R. (1991) A vertical alveolar panel (VAP) for outdoor mass cultivation of microalgae and Cyanobacteria. Bioresource Technol. 38: 153-159.

Tredici M. (2004) Mass production of microalgae: photobioreactors, pp. 178-214. In: Richmond A. (ed.) Handbook of microalgal culture: biotechnology and applied Phycology, Blackwell Science Ltd., Oxford, UK.

Watanabe Y., Joel de la Noue. And Hall D. O. (1995) Photosynthetic performance of a helical tubular photobioreactor incorporating the cyanobacterium Spirulina platensis. Biotechnol Bioeng 47: 261-269

Xiu, Z-L, Zeng, A-P, Deckwer, W-D (1998). Multiplicity and stability analysis of microorganisms in continuous culture: effects of metabolic overflow and growth inhibition, Biotechnology and Bioeng., 57:3:251-261.

We claim:

1. An isolated Chlorococcum species characterized by (i) an optimal growth temperature over 40° C., (ii) the ability to grow in a high CO₂ environment, (iii) an ability to accumulate large quantities of lutein, and (iv) an ability to assimilate large quantities of nutrients selected from the group consisting of nitrogen, phosphorous, and inorganic carbon, or progeny thereof.

2. A substantially pure culture, comprising:
(a) a growth medium; and
(b) the isolated algae of claim 1.

3. A system, comprising:
(a) a photobioreactor; and
(b) the substantially pure culture of claim 2.

4. A method for removing nutrients from wastestreams, comprising adding a waste stream to the substantially pure culture of claim 2, whereby nutrients in the waste stream are removed by the algae present in the culture.

5. A method for producing biomass, comprising
(a) culturing the algae of claim 1; and
(b) harvesting algal protein and/or biomass components from the cultured algae.

6. A method for simultaneously removing nutrients from wastestreams and producing biomass, comprising:
(a) adding a waste stream to the substantially pure culture of claim 2, whereby nutrients in the waste stream are removed by the algae present in the culture; and
(b) harvesting algal protein and/or biomass components.

7. An isolated Scenedesmus species characterized by (i) an ability to grow in a high CO₂ environment, and (ii) an ability to accumulate carotenoids selected from the group consisting of lutein, zeaxanthin, and astaxanthin, or progeny thereof.

8. A substantially pure culture, comprising:
(a) a growth medium; and
(b) the isolated algae of claim 7.

9. A system, comprising:
(a) a photobioreactor; and
(b) the substantially pure culture of claim 8.

10. A method for removing nutrients from wastestreams, comprising adding a waste stream to the substantially pure culture of claim 8, whereby nutrients in the waste stream are removed by the algae present in the culture.

11. A method for producing biomass, comprising
(a) culturing the algae of claim 7; and
(b) harvesting algal protein and/or biomass components from the cultured algae.

12. A method for simultaneously removing nutrients from wastestreams and producing biomass, comprising:
(a) adding a waste stream to the substantially pure culture of claim 8, whereby nutrients in the waste stream are removed by the algae present in the culture; and
(b) harvesting algal protein and/or biomass components.

13. An isolated Palmellococcus species, characterized by (i) an ability to grow in a high CO₂ environment, and (ii) an ability to accumulate astacene, or progeny thereof.

14. A substantially pure culture, comprising:
(a) a growth medium; and
(b) the isolated algae of claim 13.

15. A system, comprising:
(a) a photobioreactor; and
(b) the substantially pure culture of claim 14.

16. A method for removing nutrients from wastestreams, comprising adding a waste stream to the substantially pure culture of claim 14, whereby nutrients in the waste stream are removed by the algae present in the culture.

17. A method for producing biomass, comprising culturing
(a) the algae of claim 13; and
(b) harvesting algal protein and/or biomass components from the cultured algae.

18. A method for simultaneously removing nutrients from wastestreams and producing biomass, comprising:
(a) adding a waste stream to the substantially pure culture of claim 14, whereby nutrients in the waste stream are removed by the algae present in the culture; and
(b) harvesting algal protein and/or biomass components.

19. An isolated Cylindrospermopsis species, characterized by (i) an ability to assimilate large quantities of nutrients selected from the group consisting of nitrogen, phosphorous, and inorganic carbon, (ii) an ability to accumulate large quantities of protein mass, and (iii) an ability to accumulate phycobiliproteins selected from the group consisting of phycocyanin, allophycocyanin, and phycoerythrin), or progeny thereof.

20. A substantially pure culture, comprising:
(a) a growth medium; and
(b) the isolated algae of claim 19.

21. A system, comprising:
(a) a photobioreactor; and
(b) the substantially pure culture of claim 20.

22. A method for removing nutrients from wastestreams, comprising adding a waste stream to the substantially pure culture of claim 20, whereby nutrients in the waste stream are removed by the algae present in the culture.

23. A method for producing biomass, comprising
(a) culturing the algae of claim 19; and
(b) harvesting algal protein and/or biomass components from the cultured algae.

24. A method for simultaneously removing nutrients from wastestreams and producing biomass, comprising:
(a) adding a waste stream to the substantially pure culture of claim 20, whereby nutrients in the waste stream are removed by the algae present in the culture; and
(b) harvesting algal protein and/or biomass components.

25. An isolated Planktothrix species characterized by (i) an ability to assimilate large quantities of nutrients selected from the group consisting of nitrogen, phosphorous, and inorganic carbon, (ii) an ability to accumulate large quantities of protein mass, and (iii) an ability to accumulate phycobiliproteins selected from the group consisting of phycocyanin, allophycocyanin, and phycoerythrin, or progeny thereof.

26. A substantially pure culture, comprising:
(a) a growth medium; and
(b) the isolated algae of claim 25.

27. A system, comprising:
(a) a photobioreactor; and
(b) the substantially pure culture of claim 26.

28. A method for removing nutrients from wastestreams, comprising adding a waste stream to the substantially pure culture of claim 26, whereby nutrients in the waste stream are removed by the algae present in the culture.

29. A method for producing biomass, comprising
(a) culturing the algae of claim 25; and
(b) harvesting algal protein and/or biomass components from the cultured algae.

30. A method for simultaneously removing nutrients from wastestreams and producing biomass, comprising:
(a) adding a waste stream to the substantially pure culture of claim 26, whereby nutrients in the waste stream are removed by the algae present in the culture; and
(b) harvesting algal protein and/or biomass components.

31. An isolated Chlorococcum species deposited under ATCC Accession No. ______, and mutant strains derived therefrom.

32. An isolated Scenedesmus species deposited under ATCC Accession No. ______, and mutant strains derived therefrom.

33. An isolated Palmellococcus species deposited under ATCC Accession No. ______, and mutant strains derived therefrom.

34. An isolated Cylindrospermopsis species deposited under ATCC Accession No. ______, and mutant strains derived therefrom.

35. An isolated Planktothrix species deposited under ATCC Accession No. ______, and mutant strains derived therefrom.
